(19) United States
(12) Patent Application Publication
(10) Pub. No.: US 2025/0127478 A1
(43) Pub. Date: Apr. 24, 2025

(54) ACOUSTIC SENSOR AND STETHOSCOPE

(71) Applicant: Murata Manufacturing Co., Ltd., Nagaokakyo-shi (JP)

(72) Inventors: Hiroyuki KOMATSU, Nagaokakyo-shi (JP); Takatoshi KATO, Nagaokakyo-shi (JP); Hirofumi WATANABE, Nagaokakyo-shi (JP); Kohei SUGAHARA, Nagaokakyo-shi (JP)

(21) Appl. No.: 19/001,139

(22) Filed: Dec. 24, 2024

Related U.S. Application Data
(63) Continuation of application No. PCT/JP2023/023161, filed on Jun. 22, 2023.

(30) Foreign Application Priority Data
Jul. 1, 2022 (JP) 2022-106921

(51) Int. Cl. A61B 7/04; B06B 1/02; B06B 1/06; H04R 1/46; H04R 17/02

(52) U.S. Cl. CPC A61B 7/04; B06B 1/0207; B06B 1/0651; H04R 1/46; H04R 17/02; B06B 2201/40; B06B 2201/76

(57) ABSTRACT

An acoustic sensor includes a piezoelectric plate including a conductor plate with first and second surfaces, and a piezoelectric element on the second surface, a cover facing the first surface of the conductor plate, a support substrate facing the second surface of the conductor plate and the piezoelectric element, a first connector including a conductor between the piezoelectric element and the support substrate and electrically connecting the piezoelectric element and the support substrate, a first insulating structure including an insulator between the second surface of the conductor plate and the support substrate at an outer edge of the second surface of the conductor plate, and a second connector including a conductor on an outer edge of the first surface of the conductor plate and electrically connecting the conductor plate and the cover.

### CROSS REFERENCE TO RELATED APPLICATIONS

This application claims the benefit of priority to Japanese Patent Application No. 2022-106921 filed on Jul. 1, 2022 and is a Continuation Application of PCT Application No. PCT/JP2023/023161 filed on Jun. 22, 2023. The entire contents of each application are hereby incorporated herein by reference.

### BACKGROUND OF THE INVENTION

### 1. Field of the Invention

The present disclosure relates to acoustic sensors and stethoscopes.

### 2. Description of the Related Art

WO 2021/106865 A1 discloses a bioacoustic sensor including a piezoelectric plate and a stethoscope including the same. The stethoscope described in WO 2021/106865 A1 includes a diaphragm having a contact surface in contact with a living body, a piezoelectric plate that is disposed to face the diaphragm and to convert vibration of the diaphragm into an electric signal, and a vibration transmission member that is provided in a central portion of the piezoelectric plate and transmits the vibration of the diaphragm to the piezoelectric plate.

In WO 2021/106865 A1, a support member that supports each of the diaphragm and the piezoelectric plate is provided, and a cable for extracting an electric signal of the piezoelectric plate to the outside is connected. It is necessary to provide an opening through which a cable extends in a housing of the stethoscope. Therefore, in WO 2021/106865 A1, there is a possibility that the degree of freedom of the shapes and arrangement of the diaphragm and the piezoelectric plate is reduced.

### SUMMARY OF THE INVENTION

Example embodiments of the present invention provide acoustic sensors and stethoscopes each able to satisfactorily detect vibration with a simple configuration.

An acoustic sensor according to an example embodiment of the present invention includes a piezoelectric plate including a conductor plate including a first surface and a second surface opposed to the first surface, and a piezoelectric element on the second surface of the conductor plate, a cover facing the first surface of the conductor plate, a support substrate facing the second surface of the conductor plate and the piezoelectric element, a first connector including a conductor between the piezoelectric element and the support substrate and electrically connecting the piezoelectric element and the support substrate, a first insulating structure including an insulator between the second surface of the conductor plate and the support substrate at an outer edge of the second surface of the conductor plate, and a second connector including a conductor on an outer edge of the first surface of the conductor plate and electrically connects the conductor plate and the cover substrate.

A stethoscope according to an example embodiment of the present invention includes an acoustic sensor according to an example embodiment of the present invention, a chest piece in which the acoustic sensor is included, and an ear tip connected to the chest piece and configured to output, to an outside, a sound generated based on an electric signal from the piezoelectric plate of the acoustic sensor.

According to acoustic sensors and stethoscopes of the present invention, it is possible to satisfactorily detect vibration with a simple configuration.

The above and other elements, features, steps, characteristics and advantages of the present invention will become more apparent from the following detailed description of the example embodiments with reference to the attached drawings.

### BRIEF DESCRIPTION OF THE DRAWINGS

FIG. 1 is an exploded perspective view schematically illustrating a configuration of an acoustic sensor according to a first example embodiment of the present invention.

FIG. 2 is a plan view schematically illustrating a portion of the acoustic sensor according to the first example embodiment of the present invention.

FIG. 3 is a cross-sectional view taken along line III-III′ of FIG. 2.

FIG. 4 is a graph illustrating a relationship between a frequency and sensitivity of the acoustic sensor according to an example for each hardness ratio between a connector and an insulating structure.

FIG. 5 is a graph illustrating the relationship between the hardness ratio between the connector and the insulating structure and the sensitivity of the acoustic sensor according to the example.

FIG. 6 is an exploded perspective view schematically illustrating a configuration of an acoustic sensor according to a second example embodiment of the present invention.

FIG. 7 is a cross-sectional view schematically illustrating the configuration of the acoustic sensor according to the second example embodiment of the present invention.

FIG. 8 is an exploded perspective view schematically illustrating a configuration of an acoustic sensor according to a third example embodiment of the present invention.

FIG. 9 is a plan view schematically illustrating a configuration of a support substrate according to a third example embodiment of the present invention.

FIG. 10 is a plan view schematically illustrating a configuration of a back surface side of the support substrate according to the third example embodiment of the present invention.

FIG. 11 is a cross-sectional view taken along line XI-XI′ of FIG. 9.

FIG. 12 is a circuit diagram illustrating a configuration example of a detection circuit of the acoustic sensor according to the third example embodiment of the present invention.

FIG. 13 is an explanatory diagram for describing a stethoscope according to a fourth example embodiment of the present invention.

### DETAILED DESCRIPTION OF THE EXAMPLE EMBODIMENTS

Hereinafter, example embodiments of acoustic sensors and stethoscopes of the present invention will be described in detail with reference to the drawings. The present invention is not limited by the example embodiments described below. Each example embodiment is an example, and partial replacement or combination of the configurations illustrated in different example embodiments is possible. In the second and subsequent example embodiments, descriptions of matters common to the first example embodiment will be omitted, and only differences will be described. In particular, the same advantageous operations and effects by the same or similar configurations will not be sequentially described for each example embodiment.

### First Example Embodiment

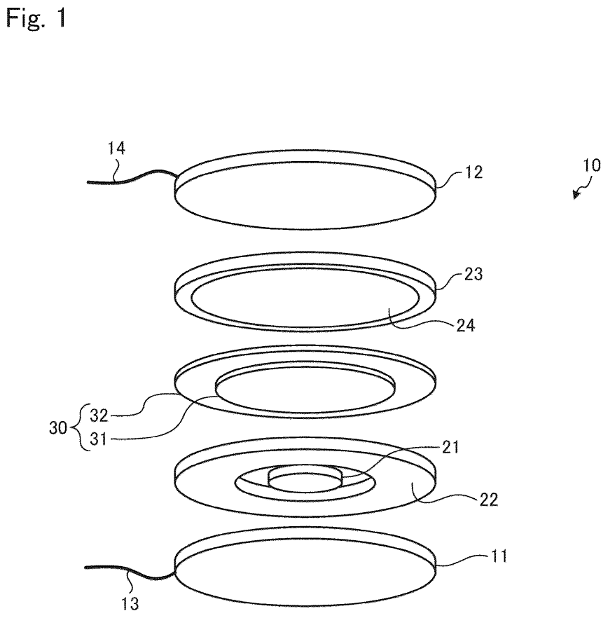

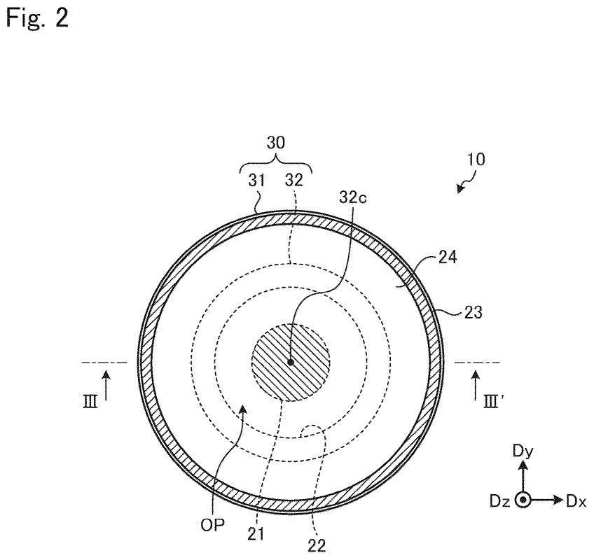

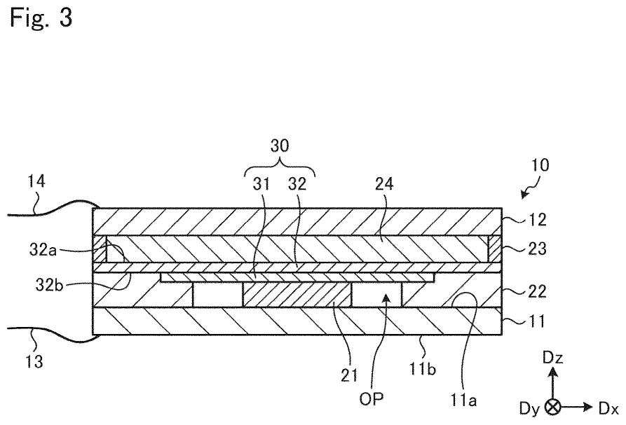

FIG. 1 is an exploded perspective view schematically illustrating a configuration of an acoustic sensor according to a first example embodiment of the present invention. FIG. 2 is a plan view schematically illustrating a portion of the acoustic sensor according to the first example embodiment. FIG. 3 is a cross-sectional view taken along line III-III′ of FIG. 2. In FIG. 2, a support substrate 11 and a cover 12 are omitted, and a first connector 21 and a second connector 23 are hatched.

As illustrated in FIGS. 1 and 3, the acoustic sensor 10 includes a piezoelectric plate 30, a support substrate 11, a cover 12, signal lines 13 and 14, a first connector 21, a first insulating structure 22, a second connector 23, and a second insulating structure 24. On the support substrate 11, the first connector 21, the first insulating structure 22, the piezoelectric plate 30, the second connector 23, the second insulating structure 24, and the cover 12 are stacked in this order.

In the following description, one direction in a plane parallel or substantially parallel to a plane including a front surface 11a of the support substrate 11 is defined as a first direction Dx. Further, a direction orthogonal or substantially orthogonal to the first direction Dx in the plane parallel or substantially parallel to the plane including the front surface 11a is defined as a second direction Dy. Furthermore, a direction orthogonal or substantially orthogonal to each of the first direction Dx and the second direction Dy is defined as a third direction Dz. The third direction Dz is a normal direction of the front surface 11a of the support substrate 11. In addition, in the present specification, a plan view indicates a positional relationship when viewed from the third direction Dz.

The piezoelectric plate 30 is a sensor element that converts vibration of an object to be detected (for example, a human) into a corresponding electric signal. Specifically, as illustrated in FIG. 3, the piezoelectric plate 30 includes a piezoelectric element 31 and a conductor plate 32. The conductor plate 32 is a plate-shaped structure made of a conductor such as, for example, a metal material, and includes a first surface 32a and a second surface 32b opposed to the first surface 32a. The piezoelectric element 31 is provided on the second surface 32b of the conductor plate 32. As the piezoelectric element 31, for example, piezoelectric ceramics such as PZT may be used.

The cover 12 is faces the first surface 32a of the conductor plate 32. The cover 12 is a plate-shaped structure made of a material having conductivity such as, for example, a metal material. The cover 12 transmits vibration of an object to be detected (not illustrated) to the piezoelectric plate 30, and is made of a material that is not substantially compressively deformed. The cover 12 may be in direct contact with the object to be detected, or may be in contact with the object to be detected via another structure, such as, for example, a protective layer.

The support substrate 11 faces the second surface 32b of the conductor plate 32 and the piezoelectric element 31. The support substrate 11 is a plate-shaped structure including a front surface 11a (surface facing the piezoelectric element 31) and a back surface 11b (surface opposed to the surface facing the piezoelectric element 31) opposite to the front surface 11a. The support substrate 11 is a conductor, and is made of, for example, a material having conductivity such as a metal material. The support substrate 11 has higher rigidity than the piezoelectric plate 30, and is configured not to be deformed by vibration of the object to be detected or to reduce or prevent deformation by vibration of the object to be detected.

The first connector 21 and the first insulating structure 22 are provided between the piezoelectric plate 30 and the support substrate 11 in the third direction Dz. Specifically, the first connector 21 is a columnar structure, and is provided between the piezoelectric element 31 and the support substrate 11. In the first connector 21, one end side in the third direction Dz is in contact with the piezoelectric element 31, and the other end side in the third direction Dz is in contact with the support substrate 11. The first connector 21 includes a conductor, and electrically connects the piezoelectric element 31 and the support substrate 11.

The first insulating structure 22 includes an insulator and is provided between the second surface 32b of the conductor plate 32 and the support substrate 11 at an outer edge of the second surface 32b of the conductor plate 32. Further, the first insulating structure 22 is also disposed so as to overlap a portion of an outer edge of the piezoelectric element 31. The first insulating structure 22 is an annular structure including an opening OP in a central portion, and the first connector 21 is provided at a position overlapping the opening OP of the first insulating structure 22. The first insulating structure 22 may be disposed so as not to overlap the outer edge of the piezoelectric element 31.

The second connector 23 and the second insulating structure 24 are provided between the piezoelectric plate 30 and the cover 12 in the third direction Dz. The second connector 23 includes a conductor, and electrically connects the conductor plate 32 and the cover 12. Specifically, the second connector 23 is an annular structure, and is provided on an outer edge of the first surface 32a of the conductor plate 32. One end side of the second connector 23 in the third direction Dz is in contact with the cover 12, and the other end side of the second connector 23 in the third direction Dz is in contact with the first surface 32a of the conductor plate 32.

The second insulating structure 24 includes an insulator, and is provided between the first surface 32a of the conductor plate 32 and the cover 12 at a central portion of the first surface 32a of the conductor plate 32. The second insulating structure 24 is provided closer to the central portion than the second connector 23, and is disposed in a region surrounded by the second connector 23.

As illustrated in FIG. 2, each structural element (piezoelectric plate 30, first connector 21, first insulating structure 22, second connector 23, and second insulating structure 24) of the acoustic sensor 10 has a circular or substantially circular shape in plan view, and is provided concentrically with respect to a center 32c of the conductor plate 32.

A diameter of the first connector 21 is smaller than a diameter of the piezoelectric element 31. An outer diameter of the first insulating structure 22 is equal or substantially equal to a diameter of the conductor plate 32. The first insulating structure 22 is provided along an entire or substantially an entire circumference of the outer edge of the second surface 32b of the conductor plate 32 and is disposed to surround the first connector 21. A diameter of the opening OP of the first insulating structure 22 (an inner diameter of the first insulating structure 22) is larger than the diameter of the first connector 21. That is, the first insulating structure 22 is spaced apart from the first connector 21 in the radial direction. Further, an inner edge defining the opening OP of the first insulating structure 22 is provided to cover an entire or substantially an entire circumference of the outer edge of the piezoelectric element 31.

The second connector 23 is provided along an entire or substantially an entire circumference of the outer edge of the first surface 32a of the conductor plate 32. An outer diameter of the second connector 23 is equal or substantially equal to the diameter of the conductor plate 32. An inner diameter of the second connector 23 is larger than the diameter of the first connector 21 and the diameter of the piezoelectric element 31. In the present example embodiment, a diameter of the second insulating structure 24 is equal or substantially equal to the inner diameter of the second connector 23. The second insulating structure 24 is provided in a region overlapping the piezoelectric element 31, the first connector 21, and the opening OP of the first insulating structure 22 in plan view. An outer periphery of the second insulating structure 24 is provided in contact with an inner periphery of the second connector 23. However, the diameter of the second insulating structure 24 may be smaller than the inner diameter of the second connector 23, and the outer periphery of the second insulating structure 24 may be spaced apart from the inner periphery of the second connector 23.

Although not illustrated in FIG. 2, as illustrated in FIGS. 1 and 3, a diameter of the support substrate 11 and a diameter of the cover 12 are equal or substantially equal to the diameter of the conductor plate 32 of the piezoelectric plate 30. However, the present disclosure is not limited thereto, and the diameter of the support substrate 11 and the diameter of the cover 12 may be larger than the diameter of the conductor plate 32 of the piezoelectric plate 30. In addition, the diameter of the support substrate 11 and the diameter of the cover 12 are the same or substantially the same, but may be different diameters.

Next, examples of materials of the first connector 21, the first insulating structure 22, the second connector 23, and the second insulating structure 24 will be described. The hardness of the first connector 21 is higher than the hardness of the first insulating structure 22. Specifically, as the material of the first connector 21, for example, carbon-based silicone, urethane foam, polybutadiene, or the like may be used. In addition, the material of the first connector 21 is, for example, preferably an independent foam structure. The hardness of the first connector 21 is Shore hardness of about A70, for example. The volume resistivity of the first connector 21 is about 1×10⁻² (Ω·m) or more and about 1×10² (Ω·m) or less, for example.

As a material of the first insulating structure 22, for example, a PET film, natural rubber (NR), chloroprene rubber (CR), polyethylene (PE), ethylene propylene rubber (EPDM), acrylic, or the like may be used. In addition, the material of the first insulating structure 22 is, for example, preferably an independent foam structure. The hardness of the first insulating structure 22 is Shore hardness of about A20, for example. The volume resistivity of the first insulating structure 22 is about 1×10⁻²⁶ (Ω·m) or more and about 1×10¹⁰ (Ω·m) or less, for example.

For the second connector 23, a material the same as or similar to the first connector 21 described above is used, and for the second insulating structure 24, a material the same as or similar to the first insulating structure 22 described above is used. That is, the hardness of the second connector 23 is higher than the hardness of the second insulating structure 24. A material example, hardness, and volume resistivity of the second connector 23 are the same as or similar to those of the first connector 21 described above, and repeated description will be omitted. In addition, a material example, hardness, and volume resistivity of the second insulating structure 24 are the same as or similar to those of the first insulating structure 22 described above, and repeated description will be omitted. The second connector 23 may be formed of the same material as the first connector 21, or may be made of a different material. In addition, the second insulating structure 24 may be made of the same material as first insulating structure 22, or may be made of a different material. For example, the second insulating structure 24 may be made of an air layer.

With such a configuration, on the first surface 32a side of the conductor plate 32, the second connector 23 harder than the second insulating structure 24 is annularly provided along an outer edge of the conductor plate 32. When the cover 12 vibrates due to vibration of the object to be detected and is repeatedly displaced in the third direction Dz, displacement of the cover 12 is mainly transmitted to an outer edge of the piezoelectric plate 30, that is, the outer edge of the conductor plate 32 via the second connector 23 harder than the second insulating structure 24.

On the second surface 32b side of the conductor plate 32, the first insulating structure 22 is provided in a region overlapping the second connector 23, and the first connector 21 harder than the first insulating structure 22 is provided in a central portion of the piezoelectric element 31. Therefore, the central portion of the piezoelectric element 31 is supported by the first insulating structure 22 to reduce or prevent displacement, and the outer edge side of the conductor plate 32 and the outer edge side of the piezoelectric element 31 are more likely to be displaced than the central portion of the piezoelectric element 31 due to elastic deformation of the first insulating structure 22. Therefore, when the displacement of the cover 12 is transmitted via the second connector 23, the outer edge side of the conductor plate 32 and the outer edge side of the piezoelectric element 31 are displaced, and the piezoelectric element 31 is bent and deformed.

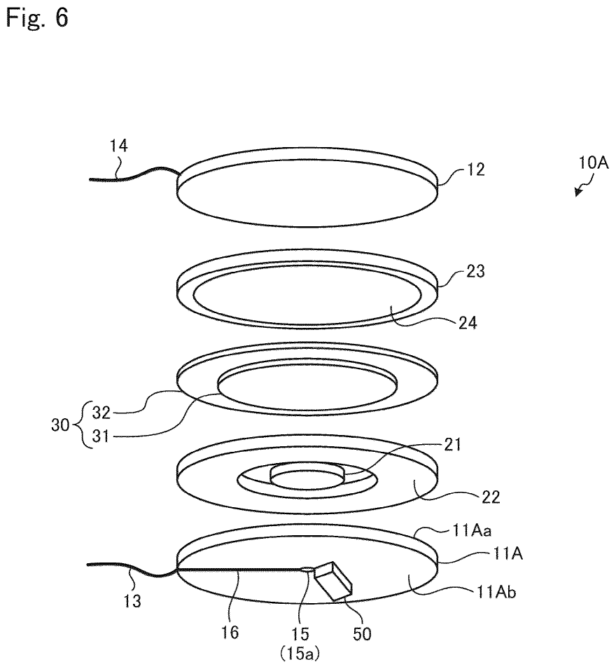

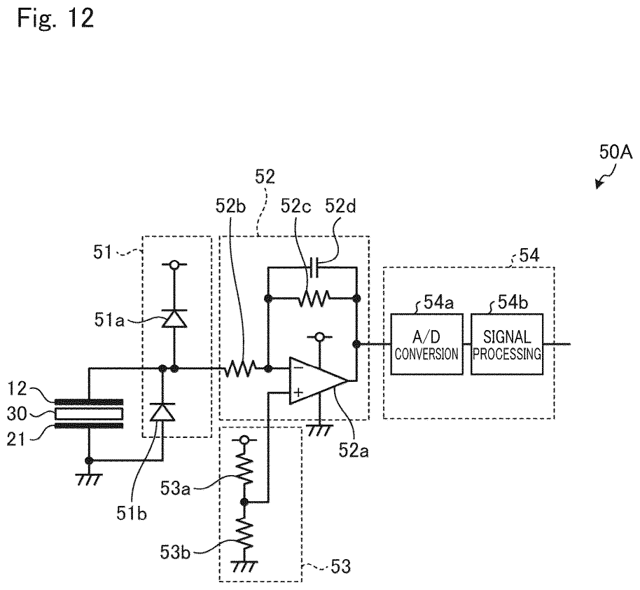

The piezoelectric element 31 outputs an electric signal corresponding to deformation. An electric signal from the piezoelectric element 31 is output to an external terminal (for example, detection circuits 50 and 50A (see FIGS. 6 and 12)) via the first connector 21, the support substrate 11, and the signal line 13. Further, the electric signal from the conductor plate 32 is output to the external terminal via the second connector 23, the cover 12, and the signal line 14.

In this manner, the first connector 21 supports a central portion of the piezoelectric plate 30, and the second connector 23 transmits the vibration of the object to be detected to the outer edge side of the piezoelectric plate 30. Furthermore, the first connector 21 and the second connector 23 also define and function as an electrical connector that outputs an electric signal from the piezoelectric plate 30 to the outside. Therefore, it is not necessary to connect a cable to extract an electric signal to the outside to the piezoelectric element 31 and the conductor plate 32. As a result, it is not necessary to provide a through-hole through which a cable passes in the support substrate 11 and the cover 12. Therefore, the acoustic sensor 10 can have a simple configuration as compared with a case where a cable is connected to the piezoelectric plate 30. As a result, the acoustic sensor 10 enables downsizing of the entire configuration including a wiring connected to the piezoelectric plate 30 and the external circuit (for example, the detection circuit 50 (see FIG. 6 and the like)).

In addition, since it is not necessary to provide a through hole for passing the cable through the support substrate 11 and the cover 12, the first surface 32a side of the conductor plate 32 can be easily sealed by the second connector 23, the second insulating structure 24, and the cover 12. Further, the second surface 32b side of the conductor plate 32 can be easily sealed by the first connector 21, the first insulating structure 22, and the support substrate 11. Therefore, the acoustic sensor 10 can have a liquid-tight structure in which both surfaces of the piezoelectric plate 30 are sealed as necessary.

As described above, the second connector 23 harder than the second insulating structure 24 is annularly provided on the first surface 32a side of the conductor plate 32. Further, on the second surface 32b side of the conductor plate 32, the first connector 21 harder than the first insulating structure 22 is provided in the central portion of the piezoelectric element 31. Thus, when the cover 12 vibrates due to the vibration of the object to be detected, the piezoelectric element 31 is easily deformed in the compression direction. Therefore, the acoustic sensor 10 can improve the resistance of the piezoelectric element 31 against excessive deflection deformation as compared with a structure in which the piezoelectric element 31 is deformed in a pulling direction.

### Example

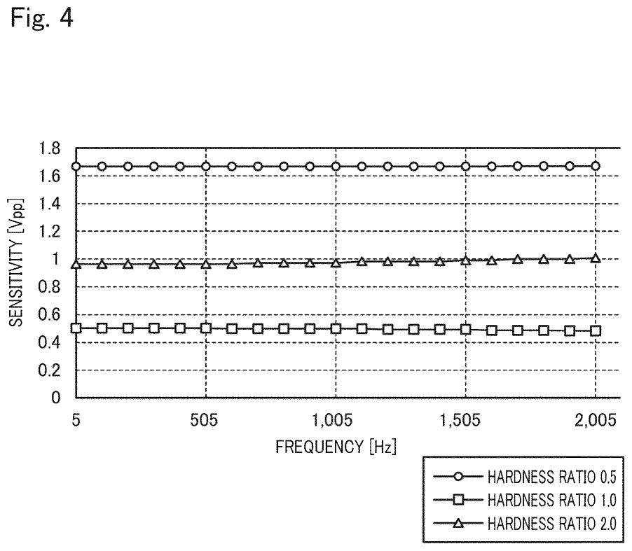

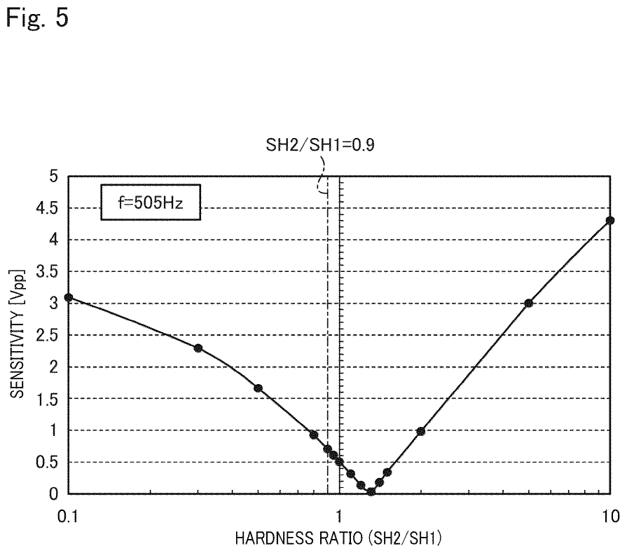

FIG. 4 is a graph illustrating the relationship between a frequency and sensitivity of the acoustic sensor according to an example for each hardness ratio between the connector and the insulating structure. FIG. 5 is a graph illustrating the relationship between the hardness ratio between the connector and the insulating structure and the sensitivity of the acoustic sensor according to the example.

FIGS. 4 and 5 illustrate simulation results of the sensitivity (output voltage) of the piezoelectric plate 30 for each frequency and each hardness ratio between the connector and the insulating structure. The frequency in FIGS. 4 and 5 is a driving frequency of displacement applied to the cover 12. Further, the hardness ratio in FIGS. 4 and 5 indicates a ratio (SH2/SH1) of the hardness (hereinafter referred to as Shore hardness SH2) of the first insulating structure 22 to the hardness (hereinafter referred to as Shore hardness SH1) of the first connector 21. The hardness and the hardness ratio of the second connector 23 and the second insulating structure 24 are the same or substantially the same as the hardness and the hardness ratio of the first connector 21 and the first insulating structure 22, respectively.

In the simulations illustrated in FIGS. 4 and 5, a diameter of the piezoelectric plate 30 (the diameter of the conductor plate 32) is set to about 15 mm, for example. The first connector 21 is made of, for example, a conductive material having a diameter of about 9 mm, a thickness of about 1 mm, and a Shore hardness of about A70. The first insulating structure 22 is made of an insulating material having an outer diameter of about 20 mm, an inner diameter of about 10 mm, a thickness of about 1 mm, and a Shore hardness of about A20, for example.

The second connector 23 is made of, for example, a conductive material having an outer diameter of about 20 mm, an inner diameter of about 10 mm, a thickness of about 1 mm, and a Shore hardness of about A70. The second insulating structure 24 is made of an insulating material having a diameter of about 9 mm, a thickness of about 1 mm, and a Shore hardness of about A20, for example.

The support substrate 11 and the cover 12 are, for example, metal plates each having a diameter of about 20 mm and a thickness of about 0.5 mm, for example.

FIG. 4 illustrates the sensitivity (output voltage) of the piezoelectric plate 30 when the hardness ratio SH2/SH1 is different as about 0.5, about 1.0, and about 2.0 in the acoustic sensor 10 according to the example. As illustrated in FIG. 4, the piezoelectric plate 30 exhibits a constant sensitivity (output voltage) to a change in frequency. Further, the piezoelectric plate 30 exhibits different sensitivities (output voltages) for each hardness ratio SH2/SH1.

FIG. 4 illustrates the sensitivity (output voltage) of the piezoelectric plate 30 when the frequency is constant at about 505 Hz. As illustrated in FIG. 5, the sensitivity (output voltage) changes according to a change in the hardness ratio SH2/SH1. When the hardness ratio SH2/SH1 is in a region of SH2/SH1>1, that is, when the hardness of the first insulating structure 22 is higher than the hardness of the first connector 21, the sensitivity (output voltage) increases as the hardness ratio SH2/SH1 increases. However, when SH2/SH1>1, the piezoelectric element 31 may be deformed in a pulling direction, leading to deterioration of durability.

In a region of SH2/SH1<1 relatively high durability is obtained, that is, in a case where the hardness of the first connector 21 is higher than the hardness of the first insulating structure 22, the sensitivity at the hardness ratio SH2/SH1=about 0.9 is about 1.4 times as high as the sensitivity at the hardness ratio SH2/SH1=about 1. As described above, for example, it is illustrated that, in the acoustic sensor 10 according to the example, by setting the hardness ratio SH2/SH1<about 0.9, the durability of the piezoelectric element 31 is improved, and satisfactory sensitivity (output voltage) is obtained.

### Second Example Embodiment

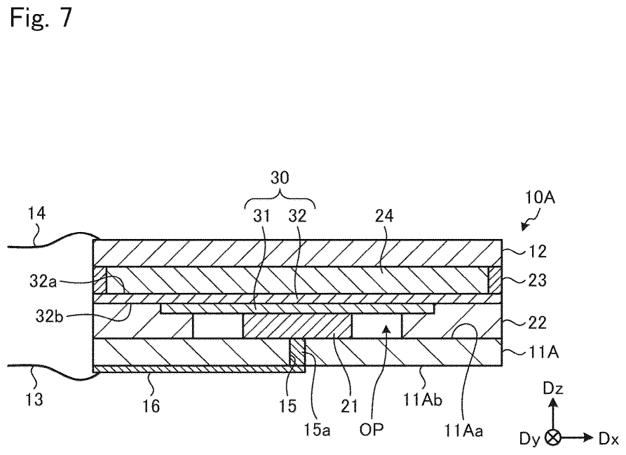

FIG. 6 is an exploded perspective view schematically illustrating a configuration of an acoustic sensor according to a second example embodiment of the present invention. FIG. 7 is a cross-sectional view schematically illustrating the configuration of the acoustic sensor according to the second example embodiment. In the second example embodiment, unlike the first example embodiment described above, a configuration in which the support substrate 11A is an insulating substrate made of an insulator will be described.

As illustrated in FIGS. 6 and 7, in the acoustic sensor 10A according to the second example embodiment, for example, a printed wiring board is used as the support substrate 11A. The support substrate 11A includes an insulating resin material as a base, and includes a conductor 15a therein. In the example illustrated in FIGS. 6 and 7, the support substrate 11A includes a contact hole 15 penetrating a front surface 11Aa (surface facing the piezoelectric element 31) and a back surface 11Ab (surface opposite to the surface facing the piezoelectric element 31). The conductor 15a is filled in the contact hole 15 to electrically connect the front surface 11Aa side and the back surface 11Ab side.

One end side of the contact hole 15 (conductor 15a) is electrically connected to the first connector 21 on the front surface 11Aa of the support substrate 11A. The other end side of the contact hole 15 (conductor 15a) is electrically connected to the connection wiring 16 at the back surface 11Ab of the support substrate 11A. Thus, the piezoelectric element 31 of the piezoelectric plate 30 is electrically connected to the back surface 11Ab side of the support substrate 11A via the first connector 21 and the contact hole 15 (conductor 15a).

As illustrated in FIG. 6, the acoustic sensor 10A of the second example embodiment includes a detection circuit 50 (not illustrated in FIG. 7) provided on the back surface 11Ab of the support substrate 11A. The detection circuit 50 includes, for example, an integrated circuit (IC), and is a circuit that performs signal processing of an electric signal from the piezoelectric plate 30.

In the acoustic sensor 10A of the present example embodiment, by using an insulating substrate as the support substrate 11A, the support substrate 11A defines and functions as a substrate that supports the piezoelectric plate 30 and also as a wiring substrate on which various wirings such as the connection wiring 16 and components such as the detection circuit 50 are mounted. Therefore, the acoustic sensor 10A miniaturizes the entire configuration including the detection circuit 50.

The support substrate 11A is not limited to the configuration including the connection wiring 16, the contact hole 15, and the conductor 15a, and may include a wiring provided in an inner layer, a plurality of contact holes, and a conductor. In addition, the back surface 11Ab of the support substrate 11A is not limited to the detection circuit 50, and other mounting components and circuits may be provided.

### Third Example Embodiment

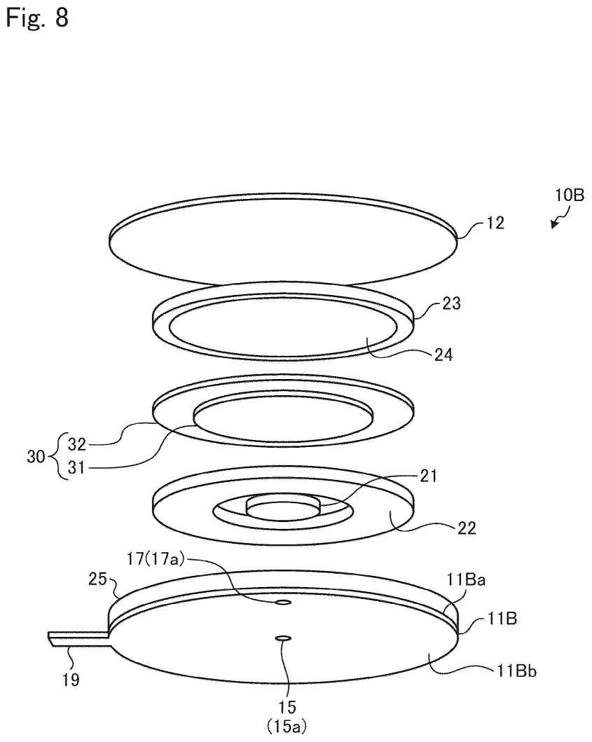

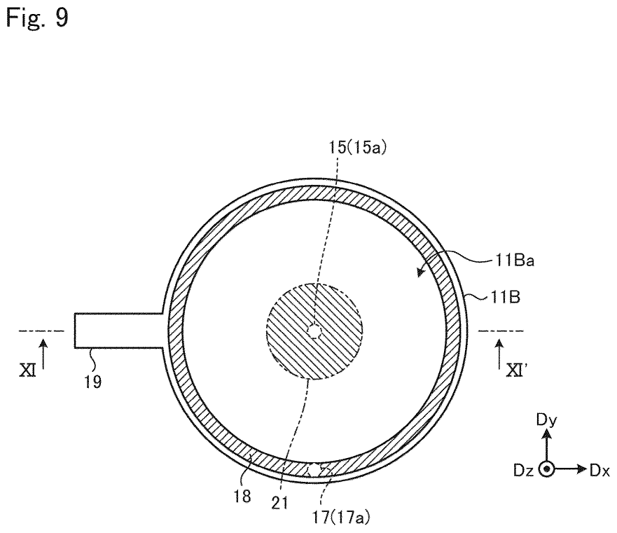

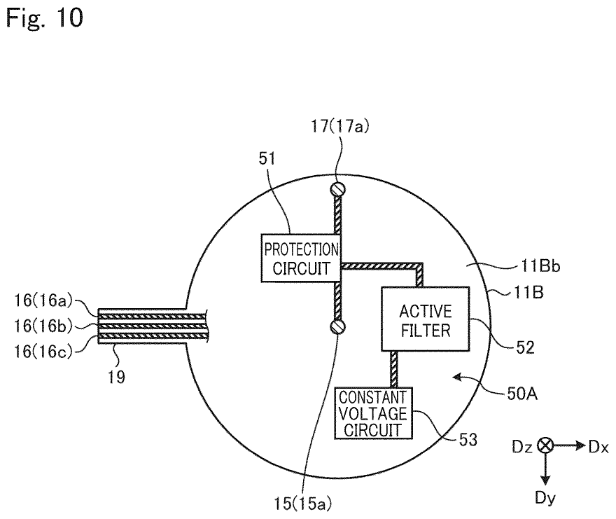

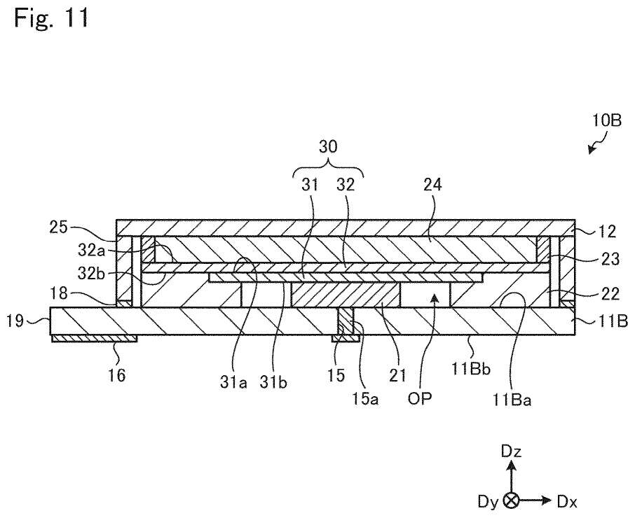

FIG. 8 is an exploded perspective view schematically illustrating a configuration of an acoustic sensor according to a third example embodiment of the present invention. FIG. 9 is a plan view schematically illustrating a configuration of a support substrate according to the third example embodiment. FIG. 10 is a plan view schematically illustrating a configuration of a back surface side of the support substrate according to the third example embodiment. FIG. 11 is a cross-sectional view taken along line XI-XI′ of FIG. 9.

In the third example embodiment, unlike the first example embodiment and the second example embodiment described above, a configuration in which an acoustic sensor 10B includes a third connector 25 that electrically connects a support substrate 11B and a cover 12 will be described.

As illustrated in FIGS. 8 and 11, in the acoustic sensor 10B according to the third example embodiment, the support substrate 11B and the cover 12 have a larger diameter (outer shape) than the conductor plate 32 of the piezoelectric plate 30. The support substrate 11B is an insulating substrate including an insulator as in the second example embodiment. Further, as in the second example embodiment, the piezoelectric element 31 of the piezoelectric plate 30 is electrically connected to a back surface 11Bb side of the support substrate 11B via the first connector 21 and the contact hole 15 (conductor 15a).

Further, the cover 12 is a film-shaped structure made of a material having conductivity. As in the first example embodiment and the second example embodiment described above, the conductor plate 32 of the piezoelectric plate 30 is electrically connected to the cover 12 via the second connector 23.

The third connector 25 includes a conductor, is provided outside the outer periphery of the conductor plate 32 of the piezoelectric plate 30, and electrically connects the support substrate 11B and the cover 12. More specifically, as illustrated in FIG. 11, the third connector 25 has an annular shape surrounding the piezoelectric plate 30, the first connector 21, the first insulating structure 22, the second connector 23, and the second insulating structure 24. The third connector 25 is annularly provided along an entire or substantially an entire circumference of the outer edge of the support substrate 11B. In addition, the third connector 25 is spaced away from outer peripheries of the conductor plate 32 of the piezoelectric plate 30, the first insulating structure 22, and the second connector 23 with a space.

One end side of the third connector 25 in the third direction Dz is in contact with the cover 12, and the other end side of the third connector 25 in the third direction Dz is electrically connected to a front surface 11Ba side of the support substrate 11B via a connection wiring 18.

As illustrated in FIG. 9, the connection wiring 18 is annularly provided along an entire circumference of the region overlapping the third connector 25, that is, the outer edge of the front surface 11Ba of the support substrate 11B. A contact hole 17 penetrating the front surface 11Ba and the back surface 11Bb is provided at a position overlapping the connection wiring 18 of the support substrate 11B. A conductor 17a is filled in the contact hole 17 and electrically connects the front surface 11Ba side and the back surface 11Bb side.

As in the second example embodiment described above, the first connector 21 is electrically connected to the back surface 11Bb side of the support substrate 11B via the contact hole 15 (conductor 15a) at a central portion of the support substrate 11B.

As illustrated in FIG. 10, a detection circuit 50A is provided on the back surface 11Bb of the support substrate 11B. The third connector 25 is electrically connected to the detection circuit 50A via a wiring provided in the contact hole 17 (conductor 17a) and the back surface 11Bb. Further, the first connector 21 is electrically connected to the detection circuit 50A via a wiring provided in the contact hole 15 (conductor 15a) and the back surface 11Bb. A detailed configuration example of the detection circuit 50A will be described below with reference to FIG. 12.

With the above configuration, the piezoelectric element 31 of the piezoelectric plate 30 is electrically connected to the detection circuit 50A on the back surface 11Bb of the support substrate 11B via the first connector 21 and the contact hole 15 (conductor 15a). The conductor plate 32 of the piezoelectric plate 30 is electrically connected to the detection circuit 50A on the back surface 11Bb of the support substrate 11B via the second connector 23, the cover 12, the third connector 25, the connection wiring 18, and the contact hole 17 (conductor 17a). Even in the configuration in which the third connector 25 is provided, since the cover 12 includes a deformable film-shaped structure, the vibration of the object to be detected is satisfactorily transmitted to the piezoelectric plate 30 via the cover 12 and the second connector 23.

In addition, the support substrate 11B includes a connection portion 19 protruding radially outward from an outer periphery. The detection circuit 50A is electrically connected to an external control board via a plurality of connection wirings 16 provided in the connection portion 19. The plurality of connection wirings 16 includes, for example, a ground line 16a that supplies a ground potential to the piezoelectric plate 30, a signal line 16b that outputs a signal from the piezoelectric plate 30 via the detection circuit 50A, a power supply line 16c that supplies a power supply potential to the detection circuit 50A, and the like.

FIG. 12 is a circuit diagram illustrating a configuration example of a detection circuit of the acoustic sensor according to the third example embodiment. As illustrated in FIG. 12, the detection circuit 50A includes a protection circuit 51, an active filter 52, a constant voltage circuit 53, and a signal processing circuit 54. The protection circuit 51 protects the piezoelectric plate 30 from overvoltage or the like, and includes diodes 51a and 51b electrically connected to the piezoelectric plate 30.

The active filter 52 is a filter circuit that passes a signal in a predetermined frequency range among signals from the piezoelectric plate 30, and includes an amplifier 52a, resistive elements 52b and 52c, and a capacitor 52d. In FIG. 12, the active filter 52 is configured as a low-pass filter, for example. However, the active filter 52 may be, for example, a high-pass filter or a band-pass filter. In addition, the present disclosure is not limited to the active filter 52, and a passive filter may be provided, or the amplifier 52a may be simply connected. The number of stages of the active filter 52 is not limited to one, and a plurality of stages may be connected.

The constant voltage circuit 53 is a circuit that supplies a constant voltage to the amplifier 52a of the active filter 52, and includes resistance elements 53a and 53b.

The signal processing circuit 54 is a circuit that processes a signal from the piezoelectric plate 30, and includes an A/D conversion circuit 54a and a signal processor 54b. The A/D conversion circuit 54a is a circuit that converts an analog signal from the piezoelectric plate 30 into a digital signal. The signal processor 54b is a circuit that receives a digital signal from the A/D conversion circuit 54a and performs signal processing such as amplification and filtering, for example.

In FIG. 10, the protection circuit 51, the active filter 52, and the constant voltage circuit 53 of the detection circuit 50A are provided on the back surface 11Bb of the support substrate 11B, and the signal processing circuit 54 is provided on an external control board. In this case, the signal line 16b (see FIG. 10) provided in the connection portion 19 is provided as a wiring to extract an analog signal from the active filter 52.

However, it is not limited thereto, and the entire detection circuit 50A including the signal processing circuit 54 may be provided on the back surface 11Bb of the support substrate 11B. In this case, the signal line 16b (see FIG. 10) provided in the connection portion 19 is configured not as an analog signal output but as a plurality of digital I/F signal input/output terminals. Specifically, a serial port such as SPI, I2C, USB, or UART, for example, or a parallel port is provided as the digital I/F signal input/output terminal.

Alternatively, a portion of the detection circuit 50A may be provided on the front surface 11Ba of the support substrate 11B. In addition, on the back surface 11Bb of the support substrate 11B, an accompanying circuit or component for stable operation of the circuit, such as, for example, a temperature sensor for temperature compensation may be mounted.

As described above, in the acoustic sensor 10B according to the third example embodiment, the cover 12 is electrically connected to the support substrate 11B via the third connector 25. Therefore, the detection circuit 50A and the various wirings can be collectively provided on the support substrate 11B, and the entire acoustic sensor 10B including the detection circuit 50A and the various wirings can be downsized.

### Fourth Example Embodiment

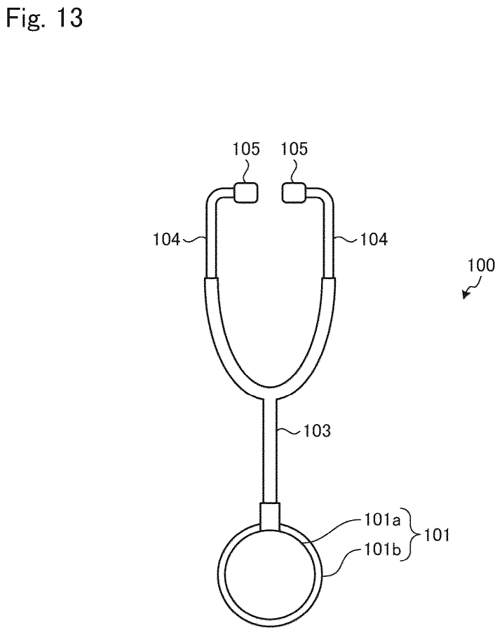

The acoustic sensors 10, 10A, and 10B of the above-described example embodiments can be used for, for example, a digital stethoscope. FIG. 13 is an explanatory diagram for describing a stethoscope according to a fourth example embodiment of the present invention. As illustrated in FIG. 13, the stethoscope 100 according to the fourth example embodiment includes a chest piece 101, a Y-shaped tube 103, two auditory tubes 104, and two ear tips 105.

The chest piece 101 includes a housing 101a and a contact portion 101b. The acoustic sensors 10, 10A, and 10B are incorporated inside the housing 101a. The contact portion 101b comes into contact with a living body (for example, human), and is configured to transmit vibration of the living body to the cover 12 (see FIG. 1 and the like). Although not illustrated, the chest piece 101 of the stethoscope 100 includes a signal processing circuit, an amplifier, a speaker, and the like that convert electric signals from the piezoelectric plates 30 of the acoustic sensors 10, 10A, and 10B into sounds. In addition, the stethoscope 100 may include a wireless communication module that transmits an electric signal from the piezoelectric plate 30 of the acoustic sensors 10, 10A, and 10B to the outside as necessary.

The Y-shaped tube 103 connects the chest piece 101 and the two auditory tubes 104. The two ear tips 105 are connected to each of the two auditory tubes 104. Sound generated based on electric signals from the piezoelectric plate 30 of the acoustic sensors 10, 10A, and 10B is output to the outside via the two ear tips 105.

Since the stethoscope 100 includes the acoustic sensor 10, 10A, or 10B, the chest piece 101 can be downsized. In addition, since the acoustic sensors 10, 10A, and 10B can have a liquid-tight structure in which both surfaces of the piezoelectric plate 30 are sealed, the stethoscope 100 including the acoustic sensor 10, 10A, or 10B can reduce or prevent damage to the piezoelectric plate 30 and the detection circuits 50 and 50A due to infiltration of moisture and antiseptic solution, for example.

In the fourth example embodiment, the stethoscope 100 including the acoustic sensors 10, 10A, or 10B has been described, but the acoustic sensors 10, 10A, and 10B are applicable to devices other than the stethoscope 100. For example, the acoustic sensors 10, 10A, and 10B may be applied to heart sound sensors that remain attached to a living body for a long time in order to monitor heart sounds.

The above-described example embodiments and modifications are merely examples, and can be appropriately changed. For example, the piezoelectric plate 30, the support substrate 11, and the cover 12 have a circular or substantially circular shape in plan view, but are not limited thereto, and the piezoelectric plate 30, the support substrate 11, and the cover 12 may have other shapes such as a quadrangular shape and a polygonal shape.

While example embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. An acoustic sensor comprising:
a piezoelectric plate including a conductor plate including a first surface and a second surface opposed to the first surface, and a piezoelectric element on the second surface of the conductor plate;
a cover facing the first surface of the conductor plate;
a support substrate facing the second surface of the conductor plate and the piezoelectric element;
a first connector including a conductor, between the piezoelectric element and the support substrate and electrically connecting the piezoelectric element and the support substrate;
a first insulating structure including an insulator between the second surface of the conductor plate and the support substrate at an outer edge of the second surface of the conductor plate; and
a second connector including a conductor on an outer edge of the first surface of the conductor plate and electrically connecting the conductor plate and the cover.

2. The acoustic sensor according to claim 1, wherein a hardness of the first connector is higher than a hardness of the first insulating structure.

3. The acoustic sensor according to claim 1, further comprising:
a second insulating structure including an insulator on the first surface of the conductor plate on a side closer to a central portion than the second connector and between the conductor plate and the cover; wherein
a hardness of the second connector is higher than a hardness of the second insulating structure.

4. The acoustic sensor according to claim 1, wherein
the first insulating structure extends along an entire or substantially an entire circumference of an outer edge of the second surface of the conductor plate; and
the second connector extends along an entire or substantially an entire circumference of an outer edge of the first surface of the conductor plate.

5. The acoustic sensor according to claim 1, wherein the support substrate includes a conductor.

6. The acoustic sensor according to claim 1, wherein
the support substrate is an insulating substrate including an insulator;
a conductor is provided inside the support substrate to electrically connect a surface of the support substrate facing the piezoelectric element and a surface of the support substrate opposite to the surface facing the piezoelectric element; and
the conductor is electrically connected to the first connector on the surface of the support substrate facing the piezoelectric element.

7. The acoustic sensor according to claim 6, wherein
the support substrate and the cover have an outer shape larger than the piezoelectric plate in plan view; and
the acoustic sensor includes a third connector including a conductor outside an outer periphery of the conductor plate of the piezoelectric plate and electrically connecting the support substrate and the cover.

8. The acoustic sensor according to claim 1, wherein the cover has a plate shape and includes a metal material.

9. The acoustic sensor according to claim 1, wherein the support substrate has a plate shape and includes a metal materials.

10. The acoustic sensor according to claim 1, wherein the first connector includes at least one of carbon-based silicone, urethane foam, or polybutadiene.

11. A stethoscope comprising:
the acoustic sensor according to claim 1;
a chest piece in which the acoustic sensor is incorporated; and
an ear tip connected to the chest piece and configured to output, to an outside, a sound generated based on an electric signal from the piezoelectric plate of the acoustic sensor.

12. The stethoscope according to claim 11, wherein a hardness of the first connector is higher than a hardness of the first insulating structure.

13. The stethoscope according to claim 11, further comprising:
a second insulating structure including an insulator on the first surface of the conductor plate on a side closer to a central portion than the second connector and between the conductor plate and the cover; wherein
a hardness of the second connector is higher than a hardness of the second insulating structure.

14. The stethoscope according to claim 11, wherein
the first insulating structure extend along an entire or substantially an entire circumference of an outer edge of the second surface of the conductor plate; and
the second connector extends along an entire or substantially an entire circumference of an outer edge of the first surface of the conductor plate.

15. The stethoscope according to claim 11, wherein the support substrate includes a conductor.

16. The stethoscope according to claim 11, wherein
the support substrate is an insulating substrate including an insulator;
a conductor is provided inside the support substrate to electrically connect a surface of the support substrate facing the piezoelectric element and a surface of the support substrate opposite to the surface facing the piezoelectric element; and
the conductor is electrically connected to the first connector on the surface of the support substrate facing the piezoelectric element.

17. The stethoscope according to claim 16, wherein
the support substrate and the cover have an outer shape larger than the piezoelectric plate in plan view; and
the acoustic sensor includes a third connector including a conductor outside an outer periphery of the conductor plate of the piezoelectric plate and electrically connecting the support substrate and the cover.

18. The stethoscope according to claim 11, wherein the cover has a plate shape and includes a metal material.

19. The stethoscope according to claim 11, wherein the support substrate has a plate shape and include a metal materials.

20. The stethoscope according to claim 11, wherein the first connector includes at least one of carbon-based silicone, urethane foam, or polybutadiene.
